(19) United States
(12) Patent Application Publication
(10) Pub. No.: US 2007/0060745 A1
(43) Pub. Date: Mar. 15, 2007

(54) Process for the preparation of fludarabine phosphate

(76) Inventors: Giovanni Cotticelli, Cernusco Sul Naviglio (IT); Barbara Verzola, Merate (IT)

Correspondence Address: PERKINS COIE LLP, P.O. BOX 2168, MENLO PARK, CA, 94026, US

(21) Appl. No.: 10/575,660

(22) PCT Filed: Oct. 13, 2004
(86) PCT No.: PCT/EP04/11494

§ 371 Date: Jun. 2, 2006

(30) Foreign Application Priority Data
Oct. 15, 2003 (IT) M12003A001994

(51) Int. Cl. C07H 19/04

(52) U.S. Cl. 536/26.3

(57) ABSTRACT

A description is given of a process for the preparation of 9-beta-D-arabinofuranosyl-2-fluoroadenine-5′-phosphate starting from 9-beta-D-arabinofuranosyl-2-fluoroadenine by reaction with a mixture composed of triethyl phosphate and phosphorus oxychloride and in accordance with a work-up which provides for the use of toluene.

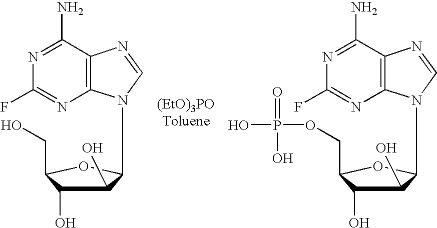

The present invention relates to a process for the preparation of 9-beta-D-arabinofuranosyl-2-fluoroadenine-5′-phosphate.

The above-mentioned compound (known under its International Non-proprietary Name as “fludarabine phosphate”), represented by formula (A): 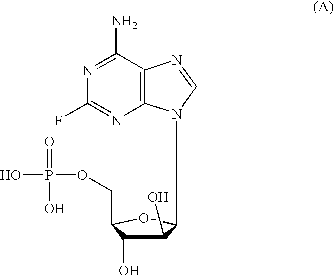
is a prodrug of 9-beta-D-arabinofuranosyl-2-fluoroadenine (known under its International Non-proprietary Name as “fludarabine”), represented by formula (B), which is used as an anti-cancer agent.

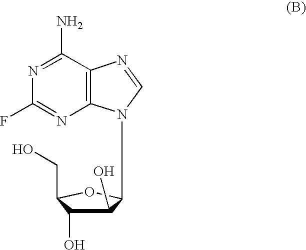

### PRIOR ART

The preparation of fludarabine phosphate has been described in various patents which are all based on fludarabine.

The document U.S. Pat. No. 4,357,324 describes a method of phosphorylation with phosphorus oxychloride and trimethyl phosphate at 0° C.; hydrolysis with water, formation of the sodium salt and subsequent conversion of the latter into the free acid.

The yields that can be obtained with the process described in that patent are modest and difficult to reproduce on an industrial scale. Moreover, the process makes use of hydroxylamine (at the extraction stage), that is to say, a compound which is potentially explosive and therefore not easy to use on a large scale.

The document U.S. Pat. No. 5,110,919 describes a phosphorylation method which provides for the use of phosphorus oxychloride and trimethyl phosphate at 0° C. The work-up consists in adding water and methylene chloride and leaving under agitation until the two phases separate. At that point, the methylene chloride is removed by decantation to give a yellowish gummy residue which is dissolved in hot water (50° C.) and left to precipitate. A “crude” product is obtained which is characterized only by a decomposition point (200-205° C.) and by purity via TLC. The recovery of a second crop by passage over resin and recrystallization of the resulting solid from water is described.

That process has the disadvantage of using a chlorinated solvent; it also makes use of a decanting operation which is difficult to carry out at an industrial level and leads to the formation of a gummy residue which, still at an industrial level, may create major agitation problems inside the reactor.

The document WO 92/00312 describes a method of phosphorylation under anhydrous conditions in which, on the one hand, the starting fludarabine is dried under vacuum and, on the other hand, the trimethyl phosphate is distilled (eliminating the head and tail fractions) in order to ensure that the system is anhydrous to the maximum extent. That process has the disadvantage of being based on the use of anhydrous reagents and starting compounds.

### DESCRIPTION OF THE INVENTION

The object of the present invention is to provide a process for the preparation of fludarabine phosphate which is free from the disadvantages of the processes of the prior art.

The invention is constituted by a process for the preparation of fludarabine phosphate in which the fludarabine is caused to react under agitation with a short-chain trialkyl phosphate and phosphorus oxychloride at a temperature of less than −5° C.; an aprotic non-polar organic solvent is then added under agitation to the mixture so obtained, still operating at a temperature of less than −5° C., with the consequent precipitation of the final product.

The starting fludarabine does not necessarily have to be anhydrous and does not have to be subjected beforehand to drying operations under vacuum; in the most advantageous embodiment of the invention, the fludarabine has a water content, measured in accordance with the Karl Fischer (K.F.) method, of not more than 0.5%.

The expression “short-chain trialkyl phosphate” means a compound of the formula (RO)₃PO wherein R is an alkyl radical having from 1 to 4 carbon atoms; the preferred short-chain trialkyl phosphates for the purposes of the present invention are trimethyl phosphate and triethyl phosphate, preferably triethyl phosphate. The short-chain trialkyl phosphate does not require previous distillation but may be used in the forms that are normally commercially available. It is preferably used in an amount of from 5 to 8 moles, more preferably from 6 to 7 moles, per mole of fludarabine (6.8 in the most advantageous embodiment) while the phosphorus oxychloride is preferably used in an amount of from 1 to 4 moles, more preferably from 2 to 3 moles, per mole of fludarabine (2.4 in the most advantageous embodiment).

The reaction is normally carried out at a temperature of less than −10° C., preferably at a temperature of from −10 to −15° C.; the duration of the reaction is normally from 24 to 48 hours, depending on the size of the reactor and the quantity of reagents.

The aprotic non-polar organic solvent is preferably a hydrocarbon solvent and, even more preferably, toluene; it is used in an amount of from 50 to 150 moles, preferably in an amount of from 100 to 110 moles, per mole of fludarabine and is preferably added dropwise at the same temperature as the reaction mixture.

The solid so obtained is simply filtered under vacuum, without then introducing decanting operations which would inevitably lead to losses of product and to operating difficulties from an industrial point of view.

The product may be subjected to purification on resin (a resin of the acid type, such as, for example, a DOWEX 50×8™ resin, is preferably used) in order to obtain a product of higher quality, and optionally to recrystallization from water at elevated temperature.

In the most advantageous embodiment of the invention, the starting fludarabine is crystallized from EtOH by suspending the fludarabine in approximately 10 volumes of EtOH; the whole is heated under reflux (78° C.) for approximately 1 hour and then cooled to ambient temperature and filtered, washing the filter cake with approximately 1 volume of EtOH. In addition to eliminating excess water, that procedure also makes it possible (without, however, having to resort to anhydrification under vacuum) to improve the quality of the fludarabine and, moreover, the method does not involve large losses of product in the mother liquors.

As will be seen from the following Examples, by operating in accordance with the process of the present invention, it is possible to obtain fludarabine phosphate with high yields and a high degree of purity without having to use anhydrous substances and/or chlorinated solvents.

### EXAMPLE 1

Fludarabine (19.5 g; 0.0683 moles) and (EtO)₃PO (79.1 ml; 0.465 moles) are introduced into a reactor cooled to −15/−20° C.

POCl₃ (15.3 ml; 0.164 moles) is added dropwise over a period of approximately 1 hour while maintaining the internal temperature at −10/−15° C. Agitation is maintained at −10/−15° C. for 48 hours; the reaction is regarded as complete when the amount of fludarabine, in the HPLC area, is less than 2%. Cold toluene (780 ml; 40 volumes) is then added over a period of approximately 1.5 hours and agitation is maintained, still at −10/−15° C., for 1-2 hours. Filtration is carried out and the filter cake is washed with toluene (20 ml). The moist solid (approximately 35 g) is suspended in H₂O (40 ml) and the pH is adjusted to 11 with 32% NaOH (approximately 20 ml). The solution is percolated into a beaker containing Dowex resin [the resin must first be activated and washed as follows: washing is effected with demineralized water until the washing liquors are colourless; acidification with 5% HCl (approximately 200 ml) is carried out and washing is effected to a neutral pH with demineralized water]. The whole is agitated for approximately 15 minutes and filtered over a septum. The resin is resuspended in H₂O (500 ml). Agitation is carried out for 15 minutes followed by filtering over a septum. This operation is repeated until no more fludarabine phosphate is present in the filtrate. The fractions containing product are reduced in volume by evaporation under vacuum (at a maximum temperature of 30-35° C.) until the desired product starts to precipitate, this product finally being filtered and dried under vacuum at 60° C. to constant weight. 10.1 g (40% yield) of a white solid having a purity greater than 97.5% are obtained. It is possible to recrystallize this solid as follows: it is suspended in 10 volumes of water and the whole is heated at 70° C. for 1 hour; the whole is filtered hot, washing the filter cake with acetone. A white solid having a purity greater than 99% is obtained.

1. A process for the preparation of fludarabine phosphate starting from fludarabine, comprising the following steps: (a) the fludarabine is caused to react with a short-chain trialkyl phosphate and phosphorus oxychloride at a temperature of less than −5° C.; (b) an aprotic non-polar organic solvent is added to the mixture so obtained with consequent precipitation of the fludarabine phosphate.

2. A process according to claim 1, characterized in that the starting fludarabine has a water content, measured in accordance with the Karl Fischer (K.F). method, of not more than 0.5%.

3. A process according to claim 1, characterized in that the short-chain trialkyl phosphate is a compound of the formula (RO)₃PO wherein R is an alkyl radical having from 1 to 4 carbon atoms.

4. A process according to claim 1, characterized in that the trialkyl phosphate is selected from trimethyl phosphate and triethyl phosphate, preferably triethyl phosphate.

5. A process according to claim 1, characterized in that the trialkyl phosphate is used in an amount of from 5 to 8 moles, preferably from 6 to 7 moles, per mole of fludarabine.

6. A process according to claim 5, characterized in that the phosphorus oxychloride is used in an amount of from 1 to 4 moles, preferably from 2 to 3 moles, per mole of fludarabine.

7. A process according to claim 1, characterized in that the aprotic non-polar organic solvent is a hydrocarbon solvent.

8. A process according to claim 7, characterized in that the aprotic non-polar organic solvent is toluene.

9. A process according to claim 1, characterized in that the aprotic non-polar organic solvent is added at a temperature of less than −5° C.

10. A process according to claim 1, characterized in that the aprotic non-polar organic solvent is used in an amount of from 50 to 150 moles, preferably in an amount of from 100 to 110 moles, per mole of fludarabine.

11. A process according to claim 1, characterized in that it is carried out at a temperature of less than −10° C., preferably at a temperature of from −10 to −15° C.
